(19) United States
(12) Patent Application Publication
(10) Pub. No.: US 2026/0207218 A1
(43) Pub. Date: Jul. 23, 2026

(54) ULTRASOUND TREATMENT TOOL

(71) Applicant: OLYMPUS MEDICAL SYSTEMS CORP., Tokyo (JP)

(72) Inventors: Kaito SATO, Tokyo (JP); Tsubasa NIIYAMA, Tokyo (JP)

(21) Appl. No.: 19/559,589

(22) Filed: Mar. 6, 2026

Related U.S. Application Data
(63) Continuation of application No. PCT/JP2024/034528, filed on Sep. 26, 2024.
(60) Provisional application No. 63/587,267, filed on Oct. 2, 2023.

(51) Int. Cl. A61B 17/32; A61B 17/00

(52) U.S. Cl. CPC A61B 17/320092; A61B 2017/00367; A61B 2017/320082; A61B 2017/320095

(57) ABSTRACT

An ultrasound treatment tool includes: a vibration transmitter configured to transmit ultrasound vibration from a proximal end toward a distal end; a tube into which the vibration transmitter is inserted; a rigid part that is made of a resin material that is harder than the tube, the rigid part being provided on an outer peripheral surface of the vibration transmitter at a node position of the ultrasound vibration, the rigid part being in contact with an inner peripheral surface of the tube; and a pipe configured to cover an outer peripheral surface of the tube. The tube is expanded by the rigid part to be in contact with an inner peripheral surface of the pipe.

### CROSS-REFERENCE TO RELATED APPLICATION

This application is a continuation of International Application No. PCT/JP2024/034528, filed on September 26, 2024 which claims the benefit of priority of U.S. Provisional Application No. 63/587,267 filed on October 2, 2023, the entire contents of which are incorporated herein by reference.

### BACKGROUND

### 1. Technical Field

The present disclosure relates to an ultrasound treatment tool.

### 2. Related Art

In the related art, there is a known ultrasound treatment tool that performs treatment on a region targeted for treatment of biological tissue (hereinafter, referred to as a treatment target) by applying ultrasound energy as treatment energy to the treatment target (for example, see International Publication Pamphlet No. WO 2016/080303).

In the ultrasound treatment tool described in Patent Literature 1, a vibration transmitter (probe), a jaw, a rubber lining (seal portion), and a pipe (sheath) described below are provided.

The vibration transmitter transmits ultrasound vibration from a proximal end toward a distal end.

The jaw is opened and closed with respect to the vibration transmitter.

The rubber lining has a circular ring shape, and is provided on an outer peripheral surface of the vibration transmitter. More specifically, the rubber lining is provided at a node position of the ultrasound vibration.

In the pipe, the vibration transmitter is inserted, and an outer peripheral surface of the rubber lining is pressure contact with the inner peripheral surface via a tube. Then, airtightness and watertightness are ensured as a result of the outer peripheral surface of the rubber lining being pressure contact with the inner peripheral surface of the pipe.

### SUMMARY

In some embodiments, an ultrasound treatment tool includes: a vibration transmitter configured to transmit ultrasound vibration from a proximal end toward a distal end; a tube into which the vibration transmitter is inserted; a rigid part that is made of a resin material that is harder than the tube, the rigid part being provided on an outer peripheral surface of the vibration transmitter at a node position of the ultrasound vibration, the rigid part being in contact with an inner peripheral surface of the tube; and a pipe configured to cover an outer peripheral surface of the tube. The tube is expanded by the rigid part to be in contact with an inner peripheral surface of the pipe.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a treatment system according to an embodiment;

FIG. 2 is a diagram explaining a configuration a distal end portion of an ultrasound treatment tool;

FIG. 3 is a diagram illustrating a configuration of a support;

FIGS. 4A and 4B are diagrams illustrating a change in shape of an inner tube caused by a third support;

FIGS. 5A and 5B are diagrams explaining a first modification of the embodiment;

FIGS. 6A and 6B are diagrams explaining a second modification of the embodiment;

FIGS. 7A and 7B are diagrams explaining a third modification of the embodiment;

FIGS. 8A and 8B are diagrams explaining a fourth modification of the embodiment;

FIGS. 9A and 9B are diagrams explaining a fifth modification of the embodiment;

FIGS. 10A and 10B are diagrams explaining a sixth modification of the embodiment;

FIGS. 11A and 11B are diagrams explaining a seventh modification of the embodiment;

FIG. 12 is a diagram explaining an eighth modification of the embodiment;

FIGS. 13A, 13B and 13C are diagrams explaining the eighth modification of the embodiment; and

FIGS. 14A, 14B and 14C are diagrams explaining a ninth modification of the embodiment.

### DETAILED DESCRIPTION

Hereinafter, modes (hereinafter, embodiments) for carrying out the present invention will be described below with reference to the drawings. Furthermore, the present invention is not limited to the embodiments described below. In addition, in description of the drawings, components that are identical to those in drawings are assigned the same reference numerals.

### Schematic configuration of treatment system

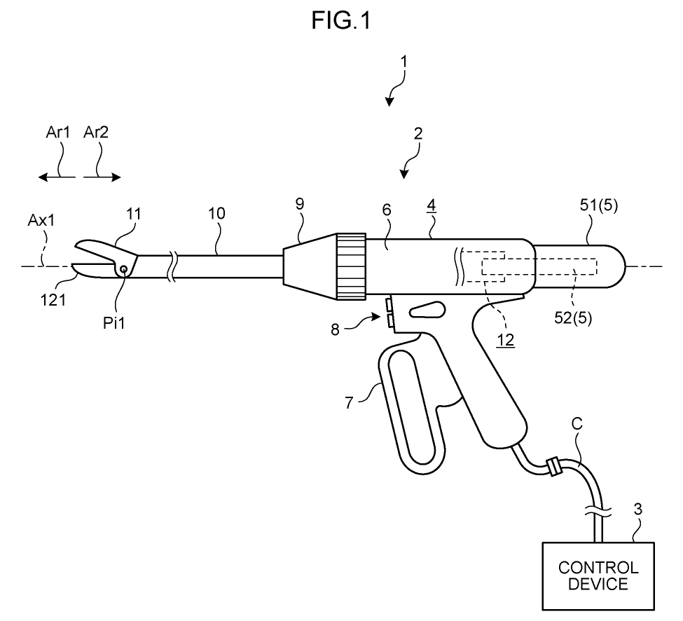

FIG. 1 is a diagram illustrating a treatment system 1 according to an embodiment.

The treatment system 1 performs treatment on a region targeted for treatment of biological tissue (hereinafter, referred to as a treatment target) by applying treatment energy to the treatment target. The treatment energy described in the present embodiment is ultrasound energy and high frequency energy. Furthermore, the treatment that is able to be performed by the treatment system 1 according to the present embodiment is treatment including coagulation (sealing) of the treatment target, incision of the treatment target, or the like. Furthermore, both of the coagulation and the incision may be performed at the same time. The treatment system 1 includes, as illustrated in FIG. 1, an ultrasound treatment tool 2 and a control device 3.

### Configuration of ultrasound treatment tool

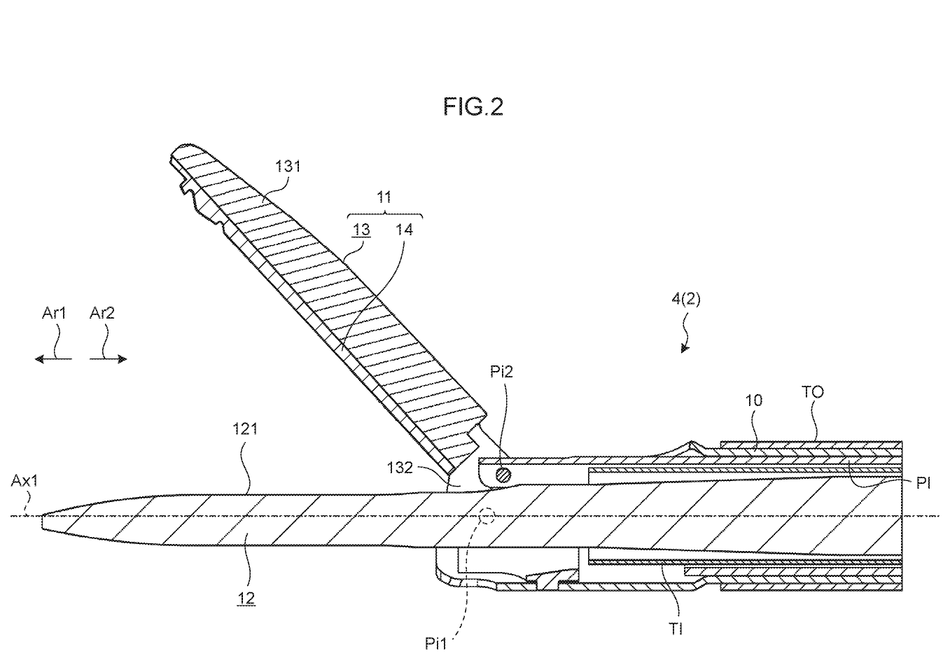

Moreover, in the following description, one of the sides along a central axis Ax1 (FIG. 1) of an outer pipe 10 is referred to as a distal end side Ar1, whereas the other of the sides is referred to as a proximal end side Ar2. In addition, a "width direction" described below is a direction perpendicular to the opening/closing direction of a jaw 11 with respect to the central axis Ax1 and a treatment portion 121, and indicates the direction perpendicular to the planes of the drawings in FIG. 1 and FIG. 2.

FIG. 2 is a diagram illustrating a configuration of a distal end portion of the ultrasound treatment tool 2. Specifically, FIG. 2 is a cross-sectional view of the distal end portion of the ultrasound treatment tool 2 obtained by cutting the distal end portion of the ultrasound treatment tool 2 by a plane that includes the central axis Ax1 of the outer pipe 10 in a state in which the jaw 11 and a vibration transmitter 12 are included in the plane.

The ultrasound treatment tool 2 is a treatment tool that performs treatment on the treatment target by applying ultrasound energy and high frequency energy to the treatment target. The ultrasound treatment tool 2 includes, as illustrated in FIG. 1, a handpiece 4 and an ultrasound transducer unit 5.

The handpiece 4 includes, as illustrated in FIG. 1 and FIG. 2, a fixed handle 6 (FIG. 1), an operation handle 7 (FIG. 1), a switch 8 (FIG. 1), a rotatable knob 9 (FIG. 1), the outer pipe 10, the jaw 11, and the vibration transmitter 12.

The fixed handle 6 is a portion that supports the entire of the ultrasound treatment tool 2, and that is gripped by an operator (user), such as an operating person.

The operation handle 7 is attached to the fixed handle 6 such that the operation handle 7 is able to move with respect to the fixed handle 6, and receives an opening and closing operation performed by the operator, such as the operating person.

The switch 8 is provided such that the switch 8 is exposed to the outside from the fixed handle 6, and receives a treatment operation performed by the operator, such as the operating person.

The rotatable knob 9 is formed to have a substantially cylindrical shape that is coaxial with the central axis Ax1, and is provided at the fixed handle 6 on the distal end side Ar1. Then, the rotatable knob 9 receives a rotation operation performed by the operator, such as the operating person. As a result of the rotation operation, the rotatable knob 9 rotates about the central axis Ax1 relative to the fixed handle 6. Furthermore, as a result of the rotation of the rotatable knob 9, the outer pipe 10, the jaw 11, and the vibration transmitter 12 also rotate about the central axis Ax1.

The outer pipe 10 has a tube shape. In the present embodiment, the outer pipe 10 is a pipe that is formed in a cylindrical shape, and that is made of an electrically conductive material, such as metal.

In the outer pipe 10, a pivot axis Pi1 (FIG. 1 and FIG. 2) that has a columnar shape, that extends in the direction perpendicular to the planes of the drawings in FIG. 1 and FIG. 2, and that is engaged with the jaw 11 and pivotally and rotatably supports the jaw 11 is fixed at the end portion of the outer pipe 10 located on the distal end side Ar1.

Then, the outer peripheral surface of the outer pipe 10 is covered by an outer tube TO (FIG. 2) having an electrical insulating property. Furthermore, an inner pipe PI (FIG. 2) that is formed in a tube shape and that moves forward and backward along the longitudinal direction of the outer pipe 10 in accordance with the opening and closing operation performed on the operation handle 7 by the operator, such as the operating person, is inserted in the interior of the outer pipe 10. The inner pipe PI corresponds to a pipe. Then, a drive axis Pi2 (FIG. 2) that has a columnar shape extending in the direction perpendicular to the planes of the drawings in FIG. 1 and FIG. 2, and that is engaged with the jaw 11 is fixed to the end portion of the inner pipe PI located on the distal end side Ar1. In the present embodiment, the drive axis Pi2 is arranged on the upper side of the pivot axis Pi1 in FIG. 2 (on the side in which an arm main body 131 is arranged with respect to the treatment portion 121).

In the present embodiment, a part of the jaw 11 is made of an electrically conductive material. The jaw 11 includes, as illustrated in FIG. 2, an arm 13 and a pad 14.

In the present embodiment, the arm 13 is made of an electrically conductive material. The arm 13 is a member that is integrally formed of, as illustrated in FIG. 2, the arm main body 131 and a pair of bearings 132.

The arm main body 131 is configured by use of a plate body having a long shape, and one of the plate surfaces is arranged in a posture in which the one of the plate surfaces faces the vibration transmitter 12.

Each of the pair of bearings 132 is provided at the end portion of the arm main body 131 located on the proximal end side Ar2, and is configured by use of a plate body that faces the width direction of the arm main body 131. Moreover, in FIG. 2, only the portion corresponding to one of the bearings 132 between the pair of bearings 132 is illustrated. The pair of bearings 132 have the same configuration, so that, in the following description, only a configuration of one of the bearings 132 will be described.

The bearing 132 is connected to the outer pipe 10 by the pivot axis Pi1. Furthermore, the bearing 132 is connected to the inner pipe PI by the drive axis Pi2.

Then, the arm 13 pivots around the pivot axis Pi1 with respect to the outer pipe 10 in conjunction with forward and backward movements of the inner pipe PI in accordance with the opening and closing operation performed on the operation handle 7 by the operator, such as the operating person. As a result of this, the jaw 11 is opened and closed with respect to the treatment portion 121 that is the end portion of the vibration transmitter 12 located on the distal end side, and enables the treatment target to be gripped between the treatment portion 121 and the jaw 11. In the present embodiment, the jaw 11 pivots around the pivot axis Pi1 in a direction closer to the treatment portion 121 in conjunction with the movement of the jaw 11 to the inner pipe PI that is located on the distal end side Ar1. In other words, the jaw 11 is closed with respect to the treatment portion 121. Furthermore, the jaw 11 pivots around the pivot axis Pi1 in a direction away from the treatment portion 121 in conjunction with the movement of the jaw 11 to the inner pipe PI that is located on the proximal end side Ar2. In other words, the jaw 11 is opened with respect to the treatment portion 121. As described above, in the present embodiment, the ultrasound treatment tool 2 is configured to have, what is called, a push close type.

The pad 14 is made of a resin material having an electrical insulating property and biological compatibility, such as, polytetrafluoroethylene (PTFE), and has a substantially rectangular parallelepiped shape along the longitudinal direction of the arm 13. Then, the pad 14 is fixed to, as illustrated in FIG. 2, the surface of the arm main body 131 located on the treatment portion 121 side, and abuts against the treatment portion 121 when the jaw 11 is closed with respect to the treatment portion 121. The pad 14 has the electrical insulating property, and accordingly has a function of preventing the arm 13 and the vibration transmitter 12 from being short circuited. Furthermore, the pad 14 has a function to prevent the treatment portion 121 that is generating ultrasound vibration from being damaged by hitting against the arm 13 when incision of the treatment target performed by using the ultrasound vibration has been completed.

Moreover, an example of the fixing method of fixing the pad 14 with respect to the arm main body 131 includes, for example, a fixing method of protruding a claw portion from the surface of the arm main body 131 located on the treatment portion 121 side and mechanically fixing by locking the pad 14 to the claw portion, a method of insert molding, or the like.

The vibration transmitter 12 is made of an electrically conductive material, and has a long shape extending along the central axis Ax1. Furthermore, the vibration transmitter 12 is, as illustrated in FIG. 2, inserted into the interior of inner pipe PI in a state in which the treatment portion 121 protrudes outside. At this time, the end portion of the vibration transmitter 12 located on the proximal end side Ar2 is, as illustrated in FIG. 1, mechanically connected to an ultrasound transducer 52 that constitutes the ultrasound transducer unit 5. Then, the vibration transmitter 12 transmits the ultrasound vibration that has been generated by the ultrasound transducer unit 5 from the end portion of the vibration transmitter 12 located on the proximal end side Ar2 to the treatment portion 121. In the present embodiment, the ultrasound vibration is longitudinal vibration that vibrates in a direction along the central axis Ax1. Furthermore, the outer peripheral surface of the vibration transmitter 12 other than the treatment portion 121 is covered by an inner tube TI (FIG. 2) that has an electrical insulating property. As the inner tube TI, it may be possible to adopt a heat shrink tube that is made of a fluororesin, such as perfluoroethylene propylene copolymer (FEP). The inner tube TI corresponds to a tube.

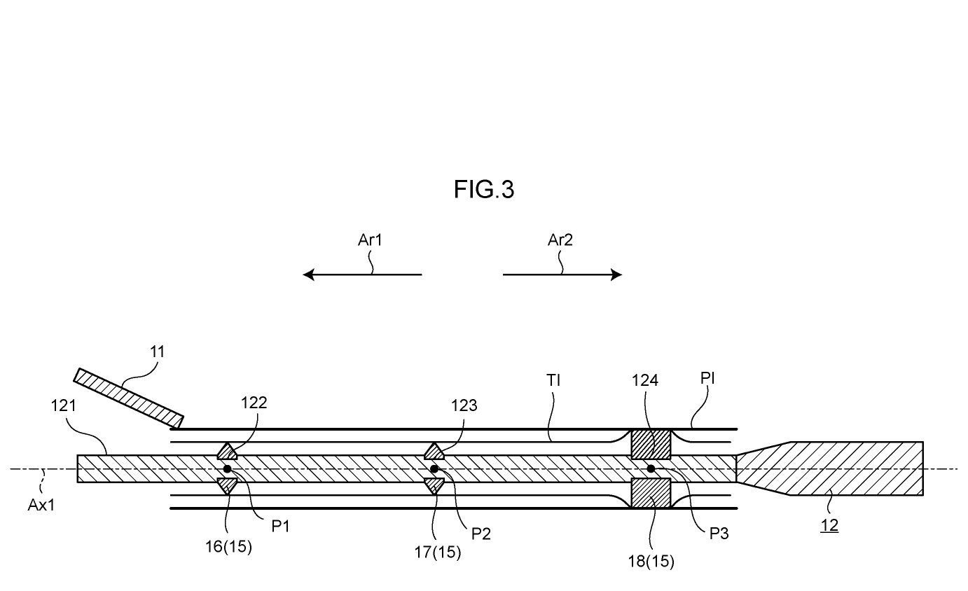

On an outer peripheral surface of the vibration transmitter 12 that has been described above, a support 15 (see FIG. 3) is provided.

Moreover, a detailed configuration of the support 15 will be explained later in a "configuration of support" that will be described later.

The ultrasound transducer unit 5 includes, as illustrated in FIG. 1, a transducer (TD) case 51 and the ultrasound transducer 52.

The TD case 51 supports the ultrasound transducer 52, and is connected to the fixed handle 6 so as to be capable of being attached to and removed from the fixed handle 6.

The ultrasound transducer 52 generates ultrasound vibration under the control of the control device 3. In the present embodiment, the ultrasound transducer 52 is constituted by a bolt-clamped Langevin-type transducer (BLT).

### Configuration of control device

The control device 3 performs overall control of the operation of the ultrasound treatment tool 2 by way of an electric cable C (FIG. 1).

Specifically, the control device 3 detects the treatment operation performed on the switch 8 by the operator, such as the operating person, by way of the electric cable C. Then, when the control device 3 has detected the treatment operation, the control device 3 applies, by way of the electric cable C, treatment energy to the treatment target that is gripped between the jaw 11 and the treatment portion 121. In other words, the control device 3 performs treatment on the treatment target.

For example, when the control device 3 applies ultrasound energy to the treatment target, the control device 3 supplies driving electrical power to the ultrasound transducer 52 by way of the electric cable C. As a result of this, the ultrasound transducer 52 generates longitudinal vibration (ultrasound vibration) that vibrates in the direction along the central axis Ax1. Furthermore, the treatment portion 121 vibrates at a desired amplitude by the longitudinal vibration. Then, the ultrasound vibration is applied from the treatment portion 121 to the treatment target that is gripped between the jaw 11 and the treatment portion 121. In other words, the ultrasound energy is applied from the treatment portion 121 to the treatment target.

Furthermore, for example, when high frequency energy is applied to the treatment target, the control device 3 supplies high frequency electrical power between the arm 13 and the vibration transmitter 12 by way of the electric cable C, the outer pipe 10, and the like. Then, when the high frequency electrical power is supplied between the arm 13 and the vibration transmitter 12, a high frequency current flows into the treatment target that is gripped between the jaw 11 and the treatment portion 121. In other words, the high frequency energy is applied to the treatment target. In other words, the arm 13 and the treatment portion 121 function as electrodes.

### Configuration of support

In the following, a configuration of the above described support 15 will be explained.

FIG. 3 is a diagram illustrating the configuration of the support 15. Specifically, FIG. 3 is a cross-sectional view obtained by cutting the jaw 11, the vibration transmitter 12, the inner pipe PI, the inner tube TI, and the support 15 by the same plane as that described above in FIG. 2.

The support 15 includes, as illustrated in FIG. 3, first to third supports 16 to 18.

In the present embodiment, each of the first and the second supports 16 and 17 is made of an elastic rubber, and has a circular ring shape into which the vibration transmitter 12 is inserted. More specifically, the first and the second supports 16 and 17 are respectively disposed at a node position P1 (FIG. 3) that is located at a position closest to the distal end side Ar1 and at a node position P2 (FIG. 3) that is located at a position second closest to the distal end side Ar1 from among the node positions of the ultrasound vibration (longitudinal vibration). Then, each of the first and the second supports 16 and 17 are in contact with the inner peripheral surface of the inner tube TI.

The third support 18 is made of a resin material that is harder than the inner tube TI, and has a circular ring shape into which the vibration transmitter 12 is inserted. Examples of the resin material include polytetrafluoroethylene (PTFE), polyether ether ketone (PEEK), polyphenylsulfone (PPSU), and polyoxymethylene (POM). More specifically, the third support 18 is disposed at a node position P3 (FIG. 3) that is located at a position closest to the proximal end side Ar2 from among the node positions of the ultrasound vibration (longitudinal vibration). The third support 18 corresponds to a rigid part.

Here, as illustrated in FIG. 3, circular ring shaped grooves 122 to 124, to which the first to the third supports 16 to 18 are respectively attached, are provided on the outer peripheral surface of the vibration transmitter 12 at the respective node positions P1 to P3.

### Change in shape of inner tube caused by third support

In the following, a change in shape of the inner tube TI caused by the above described third support 18 will be explained.

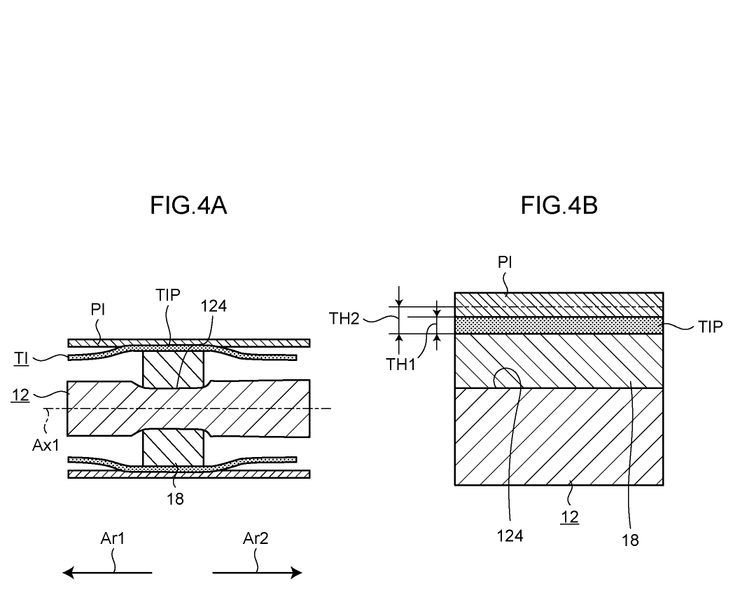

FIGS. 4A and 4B are diagrams illustrating the change in the shape of the inner tube TI caused by the third support 18. Specifically, FIG. 4A is an enlarged view of a part of FIG. 3. Furthermore, FIG. 4B is an enlarged view of an area including a part TIP of the inner tube TI indicated by FIG. 4A.

The third support 18 is, as indicated by FIG. 4A, a circular ring having a rectangular shape in cross section. Furthermore, an inner diameter dimension of the third support 18 is substantially the same as an outer diameter dimension of the groove 124. Moreover, it is preferable that the inner diameter dimension of the third support 18 is constituted to have a dimension that is smaller than the outer diameter dimension of the groove 124 in order to constitute the structure in which a gap is not formed between the third support 18 and the vibration transmitter 12. In addition, the outer diameter dimension of the third support 18 is larger than the inner diameter dimension of the inner tube TI before assembly. As a result of this, the inner tube TI is, as indicated by FIG. 4A, expanded by the third support 18 in an assembled state. Furthermore, a difference between the inner diameter dimension of the inner pipe PI and the outer diameter dimension of the third support 18 is smaller than twice the thickness dimension of the inner tube TI before assembly. As a result of this, the part TIP (FIGS. 4A and 4B) of the inner tube TI is compressed between the inner pipe PI and the third support 18. In addition, a thickness dimension TH1 of the part TIP is smaller than a thickness dimension TH2 of the other portions of the inner tube TI (FIG. 4B).

According to the above explained present embodiment, the following effects are provided.

In the ultrasound treatment tool 2 according to the present embodiment, the third support 18 is made of the resin material that is harder than that of the inner tube TI, and compresses the part TIP of the inner tube TI between the third support 18 and the inner pipe PI in a manner as described above. As a result of this, it is possible to suppress gas or a fluid that has entered the inner pipe PI from the distal end of the inner pipe PI from flowing on the proximal end side Ar2 at a place of the part TIP. Furthermore, as compared with the configuration conventionally constituted by using a rubber lining, it is possible to eliminate an operation of removing a burr, and it is thus possible to reduce a processing cost.

Therefore, with the ultrasound treatment tool 2 according to the present embodiment, it is possible to ensure airtightness and watertightness while improving a cost reduction.

### Other embodiments

In the above, descriptions of the preferred embodiments have been described, but the present invention is not limited to only the embodiments described above.

In the above described embodiment, ultrasound energy and high frequency energy are adopted as the treatment energy that is applied by the ultrasound treatment tool 2 to the treatment target, but the treatment energy is not limited to this example, and the ultrasound treatment tool 2 may use only the ultrasound energy.

In the above described embodiment, the ultrasound treatment tool 2 is configured to have, what is called, a push close type, but the configuration is not limited to this example, and the ultrasound treatment tool 2 may be configured to have, what is called, a pull close type. Specifically, the jaw 11 pivots around the pivot axis Pi1 in a direction closer to the treatment portion 121 in conjunction with the movement of the inner pipe PI to the proximal end side Ar2. In other words, the jaw 11 is closed with respect to the treatment portion 121. Furthermore, the jaw 11 pivots around the pivot axis Pi1 in a direction away from the treatment portion 121 in conjunction with the movement of the inner pipe PI to the distal end side Ar1. In other words, the jaw 11 is opened with respect to the treatment portion 121.

In the above described embodiment, the jaw 11 is opened and closed with respect to the treatment portion 121 in conjunction with the forward and backward movements of the inner pipe PI along the central axis Ax1, but the configuration is not limited to this example. It may be possible to adopt a configuration in which the jaw 11 is opened and closed with respect to the treatment portion 121 in conjunction with the forward and backward movements of the outer pipe 10 along the central axis Ax1.

In the above described embodiment, the first and the second supports 16 and 17 may also be made of a resin material, similarly to the third support 18. In a case where the first and the second supports 16 and 17 are made of the resin material in this way, as compared with a case in which the first and the second supports 16 and 17 are made of the rubber lining, it is possible to reduce an amount of deformation of the first and the second supports 16 and 17 generated at the time of closing of the jaw 11, and it is possible to suppress deflection of the vibration transmitter 12. In other words, it is possible to reliably grip the treatment target between the jaw 11 and a vibration transmitter 12.

Furthermore, in the above described embodiment, it may be possible to adopt a first to a fourth modifications that will be described below.

### First Modification

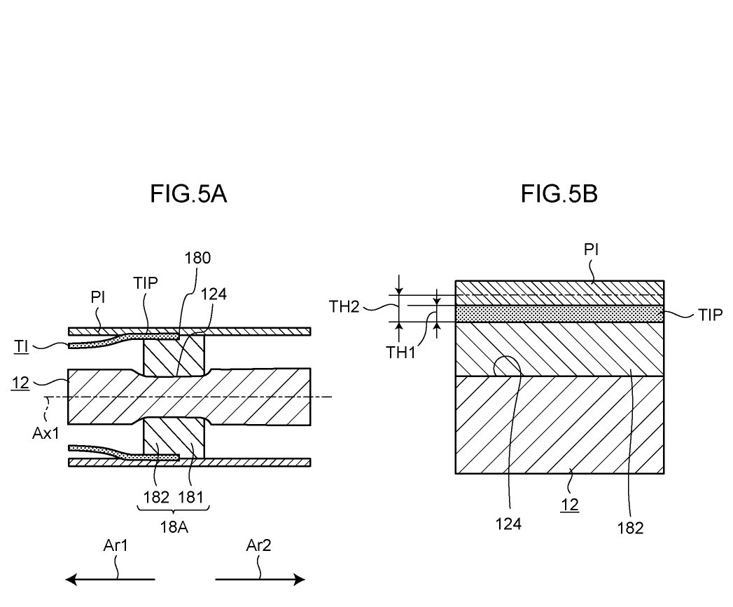

FIGS. 5A and 5B are diagrams explaining the first modification of the embodiment. Specifically, FIG. 5A is a diagram corresponding to FIG. 4A. FIG. 5B is an enlarged view of an area including the part TIP of the inner tube TI indicated by FIG. 5A.

In the above described embodiment, it may be possible to adopt a third support 18A according to the first modification illustrated in FIGS. 5A and 5B instead of adopting the third support 18.

An external shape of the third support 18A is different from that of the third support 18 that has been explained in the above described embodiment. Specifically, the third support 18A is configured by, as indicated by FIG. 5A, a large diameter portion 181 and a small diameter portion 182.

The large diameter portion 181 has a circular ring shape.

The small diameter portion 182 has a circular ring shape, and is provided in an integrated manner with the large diameter portion 181 on the distal end side Ar1 in a state of being coaxial with the large diameter portion 181. The outer diameter dimension of the small diameter portion 182 is, as illustrated in FIGS. 5A and 5B, smaller than the outer diameter dimension of the large diameter portion 181.

Then, the inner tube TI is assembled in a state in which, as indicated by FIG. 5A, the small diameter portion 182 is inserted into the interior of the inner tube TI and the end portion of the inner tube TI on the proximal end side Ar2 abuts against a stepped portion 180 located between the large diameter portion 181 and the small diameter portion 182. Here, the outer diameter dimension of the small diameter portion 182 is larger than the inner diameter dimension of the inner tube TI before assembly. As a result of this, the inner tube TI is expanded by the small diameter portion 182 in an assembled state. Furthermore, a difference between the inner diameter dimension of the inner pipe PI and the outer diameter dimension of the small diameter portion 182 is smaller than twice the thickness dimension of the inner tube TI before assembly. As a result of this, the part TIP (FIGS. 5A and 5B) of the inner tube TI is compressed between the inner pipe PI and the small diameter portion 182. In addition, the thickness dimension TH1 of the part TIP is smaller than the thickness dimension TH2 of the other portions of the inner tube TI (FIG. 5B).

According to the first modification explained above, the following effects are provided in addition to the effects similar to those described above in the embodiment.

With the third support 18A according to the first modification, it is possible to cause the stepped portion 180 to function as a positioning surface of the inner tube TI in a direction along the central axis Ax1. As a result of this, it is possible to improve ability to assemble the ultrasound treatment tool 2.

### Second Modification

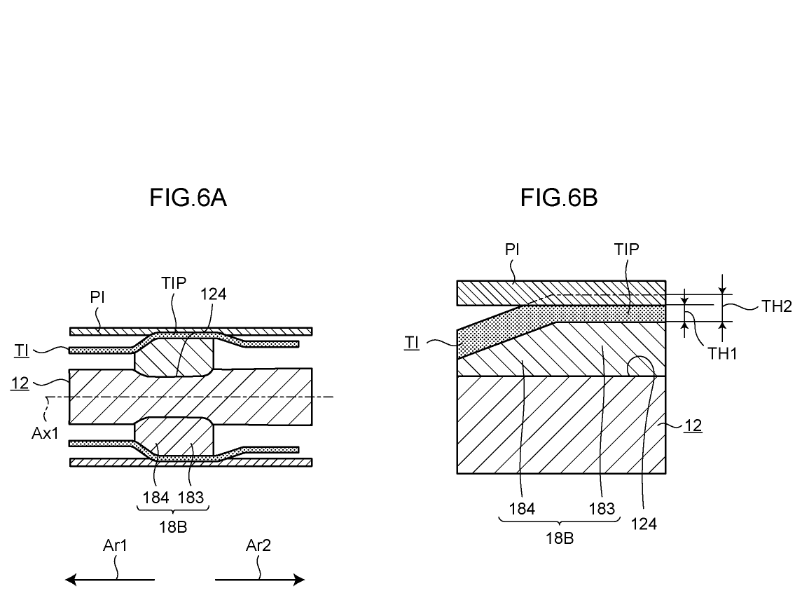

FIGS. 6A and 6B are diagrams explaining the second modification of the embodiment. Specifically, FIG. 6A is a diagram corresponding to FIG. 4A. FIG. 6B is an enlarged view of an area including the part TIP of the inner tube TI indicated by FIG. 6A.

In the above described embodiment, it may be possible to adopt a third support 18B according to the second modification illustrated in FIGS. 6A and 6B instead of adopting the third support 18.

An external shape of the third support 18B is different from that of the third support 18 that has been explained in the above described embodiment. Specifically, the third support 18B is configured by, as illustrated in FIGS. 6A and 6B, a support main body 183 and a circular truncated cone portion 184.

The support main body 183 has a circular ring shape.

The circular truncated cone portion 184 has a circular truncated cone shape as an external shape, and is provided in a posture in which the outer diameter dimension gradually decreases toward the distal end side Ar1, and is also provided in an integrated manner with the support main body 183 on the distal end side Ar1 in a state of being coaxial with the support main body 183. the largest outer diameter dimension of the circular truncated cone portion 184 is, as illustrated in FIGS. 6A and 6B, the same as the outer diameter dimension of the support main body 183. In other words, the circular truncated cone portion 184 corresponds to a tapered portion.

Then, the inner tube TI is assembled in a state in which, as indicated by FIG. 6A, the third support 18B is inserted into the interior of the inner tube TI. Here, the outer diameter dimension of each of the circular truncated cone portion 184 and the support main body 183 is larger than the inner diameter dimension of the inner tube TI before assembly. As a result of this, the inner tube TI is expanded by the third support 18B in an assembled state. Furthermore, a difference between the inner diameter dimension of the inner pipe PI and the outer diameter dimension of the support main body 183 is smaller than twice the thickness dimension of the inner tube TI before assembly. As a result of this, the part TIP (FIGS. 6A and 6B) of the inner tube TI is compressed between the inner pipe PI and the support main body 183. In addition, the thickness dimension TH1 of the part TIP is smaller than the thickness dimension TH2 of the other portions of the inner tube TI (FIG. 6B).

According to the second modification explained above, the following effects are provided in addition to the effects similar to those described above in the embodiment.

With the third support 18B according to the second modification, the outer peripheral surface of the circular truncated cone portion 184 has a tapered shape. As a result of this, the structure is constituted such that the third support 18B is easily inserted into the inner tube TI, and it is thus possible to improve ability to assemble the ultrasound treatment tool 2. Furthermore, it is possible to improve contact properties between the inner peripheral surface of the inner tube TI and the outer peripheral surface of the third support 18B, and it is thus possible to sufficiently ensure airtightness and watertightness.

### Third Modification

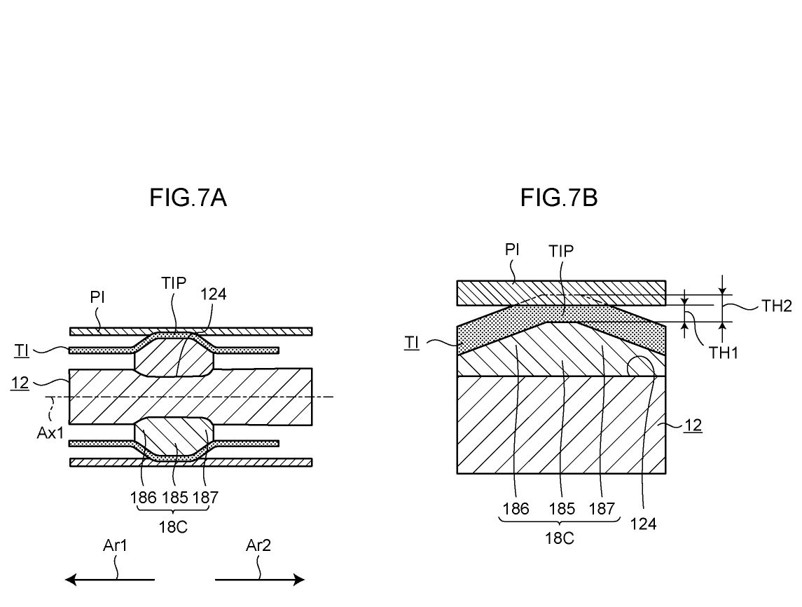

FIGS. 7A and 7B is a diagram explaining the third modification of the embodiment. Specifically, FIG. 7A is a diagram corresponding to FIG. 4A. FIG. 7B is an enlarged view of an area including the part TIP of the inner tube TI indicated by FIG. 7A.

In the above described embodiment, it may be possible to adopt a third support 18C according to the third modification illustrated in FIGS. 7A and 7B instead of adopting the third support 18.

An external shape of the third support 18C is different from that of the third support 18 that has been explained in the above described embodiment. Specifically, the third support 18C is configured by, as illustrated in FIGS. 7A and 7B, a support main body 185 and a first and a second circular truncated cone portions 186 and 187.

The support main body 185 has a circular ring shape.

The first circular truncated cone portion 186 has a circular truncated cone shape as an external shape, and is provided in a posture in which the outer diameter dimension gradually decreases toward the distal end side Ar1, and is also provided in an integrated manner with the support main body 185 on the distal end side Ar1 in a state of being coaxial with the support main body 185. In the first circular truncated cone portion 186, the largest outer diameter dimension is, as illustrated in FIGS. 7A and 7B, the same as the outer diameter dimension of the support main body 185. In other words, the first circular truncated cone portion 186 corresponds to the tapered portion.

The second circular truncated cone portion 187 has a circular truncated cone shape as an external shape, and is provided in a posture in which the outer diameter dimension gradually decreases toward the proximal end side Ar2, and is also provided in an integrated manner with the support main body 185 on the proximal end side Ar2 in a state of being coaxial with the support main body 185. In the second circular truncated cone portion 187, the largest outer diameter dimension is, as illustrated in FIGS. 7A and 7B, the same as the outer diameter dimension of the support main body 185. In other words, the second circular truncated cone portion 187 corresponds to the tapered portion.

Then, the inner tube TI is assembled in a state in which, as indicated by FIG. 7A, the third support 18C is inserted into the interior of the inner tube TI. Here, the outer diameter dimension of each of the first and the second circular truncated cone portions 186 and 187 and the support main body 185 is larger than the inner diameter dimension of the inner tube TI before assembly. As a result of this, the inner tube TI is expanded by the third support 18C in an assembled state. Furthermore, a difference between the inner diameter dimension of the inner pipe PI and the outer diameter dimension of the support main body 185 is smaller than twice the thickness dimension of the inner tube TI before assembly. As a result of this, the part TIP (FIGS. 7A and 7B) of the inner tube TI is compressed between the inner pipe PI and the support main body 185. In addition, the thickness dimension TH1 of the part TIP is smaller than the thickness dimension TH2 of the other portions of the inner tube TI (FIG. 7B).

According to the third modification explained above, the following effects are provided in addition to the effects similar to those described above in the embodiment and the second modification.

In the third modification, it is possible to shorten the length dimension of the part TIP of the inner tube TI in the direction along the central axis Ax1. As a result of this, it is possible to reduce sliding resistance generated when moving the inner pipe PI forward and backward in a direction along the central axis Ax1 in order to open and close the jaw 11 with respect to the treatment portion 121.

### Fourth Modification

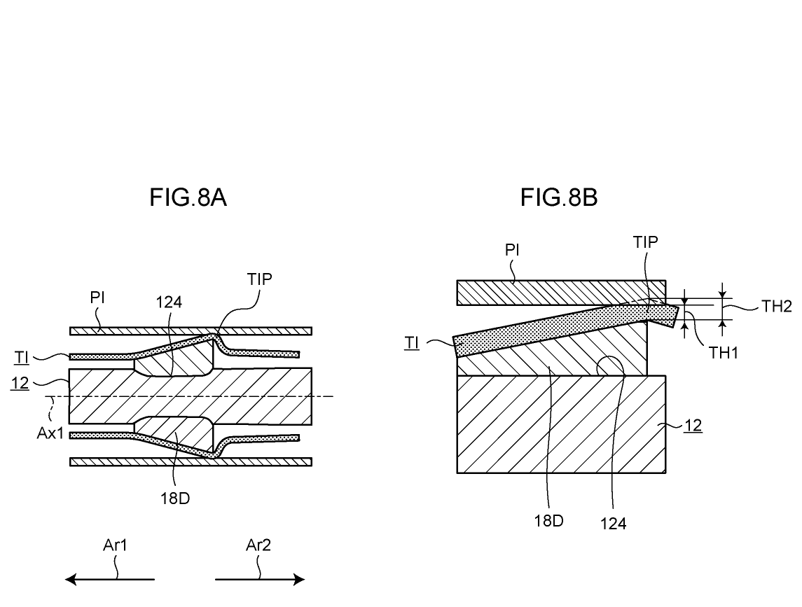

FIGS. 8A and 8B are diagrams explaining the fourth modification of the embodiment. Specifically, FIG. 8A is a diagram corresponding to FIG. 4A. FIG. 8B is an enlarged view of an area including the part TIP of the inner tube TI indicated by FIG. 8A.

In the above described embodiment, it may be possible to adopt a third support 18D according to the fourth modification illustrated in FIGS. 8A and 8B instead of adopting the third support 18.

An external shape of the third support 18D is different from that of the third support 18 that has been explained in the above described embodiment. Specifically, the third support 18D is configured such that, as illustrated in FIGS. 8A and 8B, the outer diameter dimension gradually decreases toward the distal end side Ar1. In other words, the third support 18D corresponds to the tapered portion.

Then, the inner tube TI is assembled in a state in which, as indicated by FIG. 8A, the third support 18D is inserted into the interior of the inner tube TI. Here, the outer diameter dimension of the third support 18D is larger than the inner diameter dimension of the inner tube TI before assembly. As a result of this, the inner tube TI is expanded by the third support 18D in an assembled state. Furthermore, a difference between the inner diameter dimension of the inner pipe PI and the outer diameter dimension of the end portion of the third support 18D located on the proximal end side Ar2 is smaller than twice the thickness dimension of the inner tube TI before assembly. As a result of this, the part TIP (FIGS. 8A and 8B) of the inner tube TI is compressed between the inner pipe PI and the end portion of the third support 18D located on the proximal end side Ar2. In addition, the thickness dimension TH1 of the part TIP is smaller than the thickness dimension TH2 of the other portions of the inner tube TI (FIG. 8B).

Even in a case where the configuration of the third support 18D according to the fourth modification explained above is used, the same effects similar to those described above in the embodiment and the third modification are provided.

### Fifth Modification

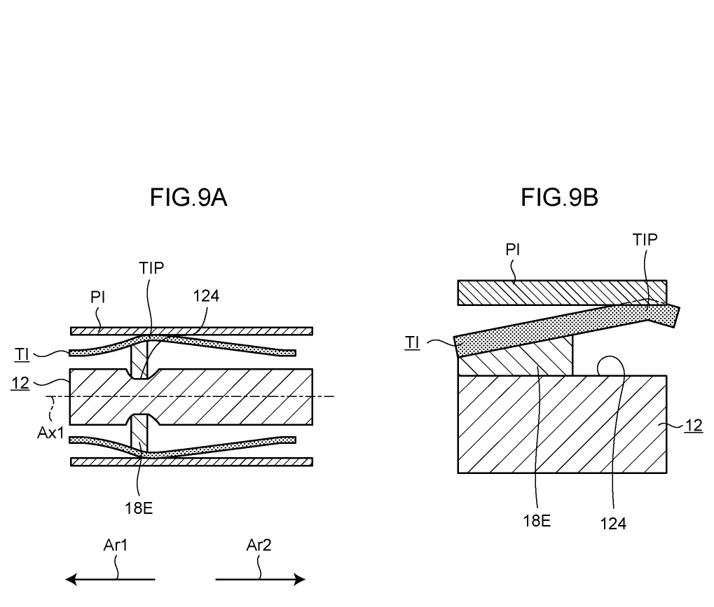

FIG. 9A and 9B are diagrams explaining the fifth modification according to the embodiment. Specifically, FIG. 9A is a diagram corresponding to FIG. 4A. FIG. 9B is an enlarged view of an area including the part TIP of the inner tube TI indicated by FIG. 9A.

In the above described embodiment, it may be possible to adopt a third support 18E according to the fifth modification illustrated in FIGS. 9A and 9B instead of adopting the third support 18.

An external shape of the third support 18E is different from that of the third support 18 that has been explained in the above described embodiment. Specifically, the third support 18E is configured such that, as illustrated in FIGS. 9A and 9B, the outer diameter dimension gradually decreases toward the distal end side Ar1. In other words, the third support 18E corresponds to the tapered portion.

Then, the inner tube TI is assembled in a state in which, as indicated by FIG. 9A, the third support 18E is inserted into the interior of the inner tube TI. Here, the outer diameter dimension of the third support 18E is larger than the inner diameter dimension of the inner tube TI before assembly. As a result of this, the inner tube TI is expanded by the third support 18E in an assembled state. Furthermore, a difference between the inner diameter dimension of the inner pipe PI and the outer diameter dimension of the end portion of the third support 18E located on the proximal end side Ar2 is smaller than twice the thickness dimension of the inner tube TI before assembly. As a result of this, the inner tube TI is not compressed between the inner pipe PI and the end portion of the third support 18E located on the proximal end side Ar2. Then, the part TIP of the inner tube TI abuts against the inner peripheral surface of the inner pipe PI at a position away from the third support 18E toward the proximal end side Ar2 (FIG. 9B).

According to the fifth modification explained above, the following effects are provided in addition to the effects similar to those described above in the embodiment.

In the fifth modification, the inner tube TI is not compressed between the inner pipe PI and the third support 18E. Then, the part TIP of the inner tube TI is contact with the inner peripheral surface of the inner pipe PI at the position away from the third support 18E toward the proximal end side Ar2. As a result of this, it is possible to allow the part TIP of the inner tube TI to be in contact with the inner peripheral surface of the inner pipe PI at a pressure that is sufficient to ensure watertightness and airtightness. Furthermore, it is possible to reduce sliding resistance generated when moving the inner pipe PI forward and backward in a direction along the central axis Ax1 in order to open and close the jaw 11 with respect to the treatment portion 121.

### Sixth Modification

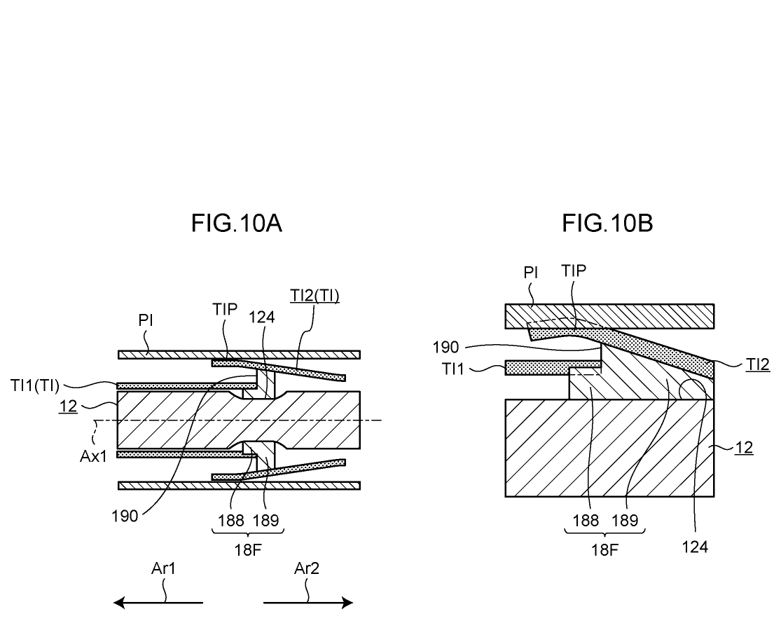

FIGS. 10A and 10B are diagrams explaining the sixth modification of the embodiment. Specifically, FIG. 10A is a diagram corresponding to FIG. 4A. FIG. 10B is an enlarged view of an area including the part TIP of the inner tube TI indicated by FIG. 10A.

In the above described embodiment, it may be possible to use a third support 18F according to the sixth modification illustrated in FIGS. 10A and 10B instead of adopting the third support 18. Furthermore, in the sixth modification, the inner tube TI is divided into two portions, as indicated by FIG. 10A, in a direction along the central axis Ax1. In the following description, between the two portions, the inner tube TI that is located on the distal end side Ar1 is referred to as a first inner tube TI1, and the inner tube TI that is located on the proximal end side Ar2 is referred to as a second inner tube TI2.

An external shape of the third support 18F is different from that of the third support 18 that has been explained in the above described embodiment. Specifically, the third support 18F is configured by, as illustrated in FIGS. 10A and 10B, a circular ring portion 188 and a circular truncated cone portion 189.

The circular ring portion 188 has a circular ring shape. The outer diameter dimension of the circular ring portion 188 is, as indicated by FIG. 10A, substantially the same as the thickness dimension of the first inner tube TI1 before assembly.

The circular truncated cone portion 189 has a circular truncated cone shape as an external shape, and is provided in a posture in which the outer diameter dimension gradually decreases toward the proximal end side Ar2, and is also provided in an integrated manner with the circular ring portion 188 on the proximal end side Ar2 in a state of being coaxial with the circular ring portion 188. In the circular truncated cone portion 189, the largest outer diameter dimension is, as illustrated in FIGS. 10A and 10B, larger than the outer diameter dimension of the circular ring portion 188. In other words, the circular truncated cone portion 189 corresponds to the tapered portion.

Then, the first inner tube TI1 is assembled in a state in which, as illustrated in FIGS. 10A and 10B, the circular ring portion 188 is inserted into the interior of the first inner tube TI1 and the end portion of the first inner tube TI1 on the proximal end side Ar2 abuts against a stepped portion 190 disposed between the circular ring portion 188 and the circular truncated cone portion 189.

On the other hand, the second inner tube TI2 is assembled in a state in which, as illustrated in FIGS. 10A and 10B, the circular truncated cone portion 189 is inserted into the interior of the second inner tube TI2. Here, the outer diameter dimension of the circular truncated cone portion 189 is larger than the inner diameter dimension of the second inner tube TI2 before assembly. As a result of this, the second inner tube TI2 is expanded by the circular truncated cone portion 189 toward the distal end side Ar1 in an assembled state. Furthermore, a difference between the inner diameter dimension of the inner pipe PI and the outer diameter dimension of the end portion of the circular truncated cone portion 189 located on the distal end side Ar1 is greater than twice the thickness dimension of the second inner tube TI2 before assembly. As a result of this, the second inner tube TI2 is not compressed between the inner pipe PI and the end portion of the circular truncated cone portion 189 located on the distal end side Ar1. In addition, the part TIP of the second inner tube TI2 is contact with the inner peripheral surface of the inner pipe PI at a position away from the circular truncated cone portion 189 toward the distal end side Ar1 (FIG. 10B).

According to the sixth modification explained above, the following effects are provided in addition to the effects similar to those described above in the embodiment and the fifth modification.

In the sixth modification, the second inner tube TI2 is expanded by the circular truncated cone portion 189 toward the distal end side Ar1. Furthermore, the part TIP of the second inner tube TI2 is contact with the inner peripheral surface of the inner pipe PI at a position away from the circular truncated cone portion 189 toward the distal end side Ar1. As a result of this, the structure is constituted such that, for example, in a surgical operation performed by using a pneumoperitoneum apparatus, when treatment is performed while pressurizing an abdominal cavity, in a case where high-pressure gas enters the interior of the inner pipe PI from the distal end of the inner pipe PI toward the proximal end side Ar2 inside the abdominal cavity, the part TIP of the second inner tube TI2 is pressure contact with the inner peripheral surface of the inner pipe PI by the high-pressure gas. As a result of this, it is possible to effectively ensure airtightness and watertightness.

### Seventh Modification

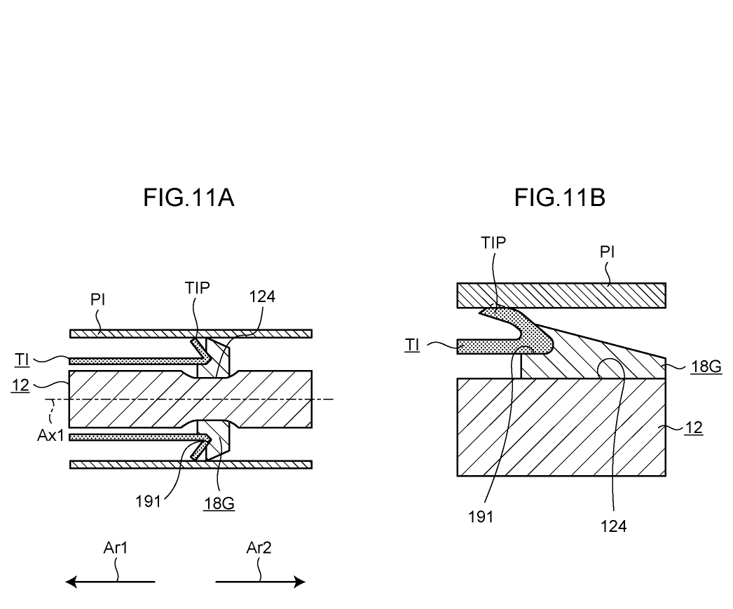

FIGS. 11A and 11B are diagrams explaining the seventh modification of the embodiment. Specifically, FIG. 11A is a diagram corresponding to FIG. 4A. FIG. 11B is an enlarged view of an area including the part TIP of the inner tube TI indicated by FIG. 11A.

In the above described embodiment, it may be possible to adopt a third support 18G according to the seventh modification illustrated in FIGS. 11A and 11B instead of adopting the third support 18.

An external shape of the third support 18G is different from that of the third support 18 that has been explained in the above described embodiment. Specifically, the third support 18G is, as illustrated in FIGS. 11A and 11B, recessed from the distal end side Ar1 toward the proximal end side Ar2, and a turn over portion 191 that turns up the end portion of the inner tube TI located on the proximal end side Ar2 is provided.

Then, the inner tube TI is assembled in a state in which, as illustrated in FIGS. 11A and 11B, the end portion of the proximal end side Ar2 is turned up by the turn over portion 191. Then, in the inner tube TI, the part TIP that has been turned up is contact with the inner peripheral surface of the inner pipe PI at a position away from the third support 18G toward the distal end side Ar1 (FIG. 11B).

Even in a case where the configuration of the third support 18G according to the seventh modification explained above is used, the same effects similar to those described above in the embodiment and the sixth modification.

### Eighth Modification

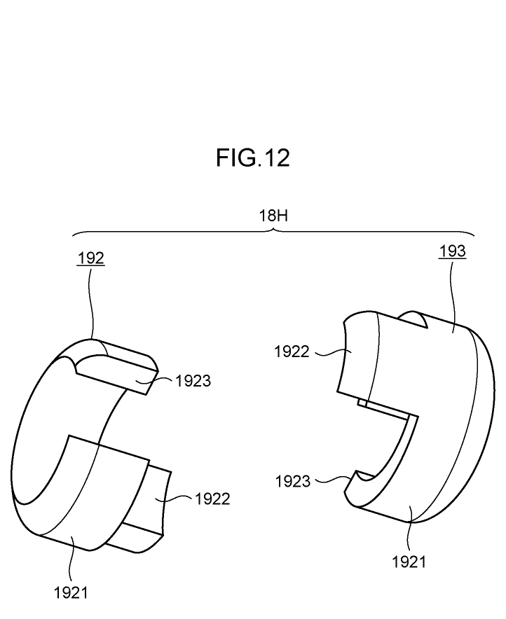

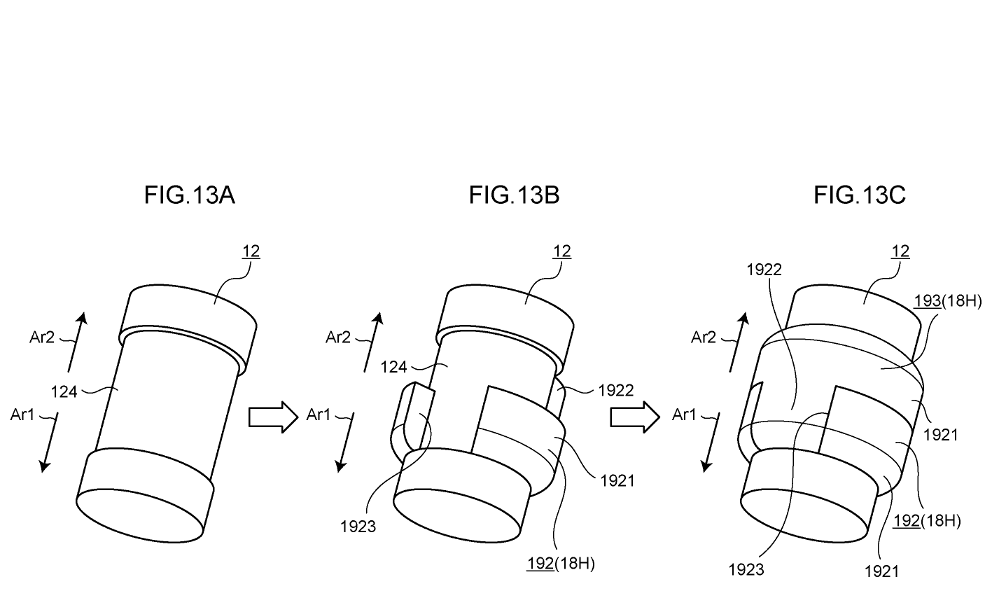

FIG. 12 and FIGS. 13A, 13B and 13C are diagrams each explaining the eighth modification of the embodiment. Specifically, FIG. 12 is an exploded perspective view illustrating a third support 18H according to the eighth modification. FIGS. 13A, 13B and 13C are diagrams illustrating an assembly order of the third support 18H with respect to the vibration transmitter 12.

In the above described embodiment, as the third support 18, it may be possible to adopt a configuration of the third support 18H according to the eighth modification illustrated in FIG. 12 and FIGS. 13A, 13B and 13C.

The third support 18H is configured by, as illustrated in FIG. 12 and FIGS. 13A, 13B and 13C, a first and a second C rings 192 and 193.

Each of the first and the second C rings 192 and 193 is made of the same resin material as that used for the third support 18 that has been described above in the embodiment, and has the same shape. Therefore, in the following, only the configuration of the first C ring 192 will be described, and regarding the second C ring 193, components that are the same as those included in the first C ring 192 are assigned the same reference numerals and descriptions thereof in detail will be omitted.

The first C ring 192 includes, as illustrated in FIG. 12, a C ring main body 1921 and a protrusion 1922.

The C ring main body 1921 is a member that has a C-shape that has been formed as a result of a notch 1923 being formed at a part of circumferential direction of a circular ring that has substantially the same inner diameter dimension as the outer diameter dimension of the groove 124.

The protrusion 1922 is a portion that protrudes along the central axis of the C ring main body 1921 from a position that is rotationally symmetrical at 180° about the central axis of the C ring main body 1921 with respect to a place in which the notch 1923 is provided on the C ring main body 1921.

Then, the third support 18H is assembled, as described below, in the groove 124.

Specifically, first, a worker arranges the vibration transmitter 12 on an inner side of the C ring main body 1921 formed in a C-shape while pushing out both end portions of the circumferential direction of the C ring main body 1921 included in the first C ring 192 (both edge portions of the notch 1923) in a direction away from each other (FIG. 13B). Furthermore, the worker arranges, in a similar manner, the vibration transmitter 12 on an inner side of the C ring main body 1921 formed in the C-shape while pushing out both end portions of the circumferential direction of the C ring main body 1921 included in the second C ring 193 (both edge portions of the notch 1923) in a direction away from each other in a state in which the protrusion 1922 included in the second C ring 193 fits into the notch 1923 included in the first C ring 192 (FIG. 13C). In other words, the first and the second C rings 192 and 193 are attached to the vibration transmitter 12 by using, what is called, a snap-fit method.

According to the eighth modification explained above, the following effects are provided in addition to the effects similar to those described above in the embodiment.

The third support 18H according to the eighth modification is configured by the first and the second C rings 192 and 193 that has been described above. As a result of this, it is possible to attach the first and the second C rings 192 and 193 to the vibration transmitter 12 by using, what is called, the snap-fit method, and it is possible to improve ability to assemble the ultrasound treatment tool 2. Furthermore, it is possible to reduce assembly time, and it is thus possible to improve a cost reduction of the ultrasound treatment tool 2. In addition, the third support 18H is made of a resin material, so that it is possible to narrow down a gap between the first and the second C rings 192 and 193 located at the boundary portion and a path through which a fluid flows can be made long and complex in shape, and as a result of this, it is possible to sufficiently ensure watertightness. In particular, a part of the boundary portion intersects (perpendicular to) a direction along the central axis Ax1, so that it is possible to effectively suppress the fluid from entering. Furthermore, by manufacturing the third support 18H that is made of the resin material by a 3D printer, it is possible to manufacture the third support 18H at low cost.

### Ninth Modification

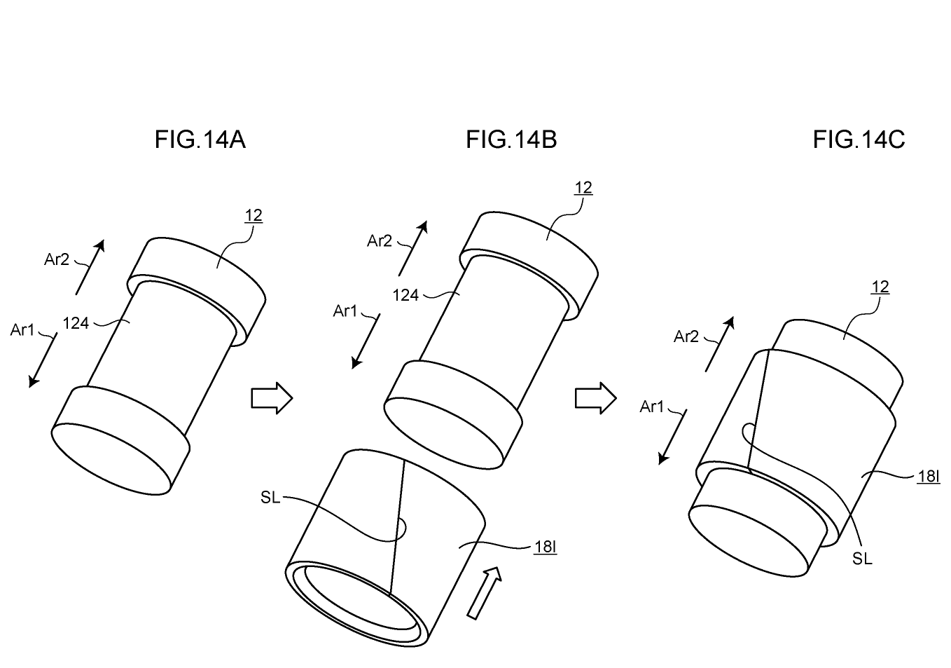

FIGS. 14A, 14B and 14C are diagrams explaining the ninth modification of the embodiment. Specifically, FIGS. 14A, 14B and 14C are diagrams illustrating an assembly order of a third support 18I according to the ninth modification with respect to the vibration transmitter 12.

In the above described embodiment, it may be possible to adopt a configuration of the third support 18I according to the ninth modification illustrated in FIGS. 14A, 14B and 14C as the third support 18.

The third support 18I is made of the same resin material as that used for the third support 18 that has been described above in the embodiment. Specifically, the third support 18I has, as indicated by FIG. 14B, substantially the same inner diameter dimension as the outer diameter dimension of the groove 124, and has a circular ring shape into which the vibration transmitter 12 is inserted. Then, in the third support 18I, a slit SL that extends from one end toward the other end of the third support 18I in the direction along the central axis Ax1 is provided (FIG. 14B).

Then, the third support 18I is assembled, as described below, in the groove 124.

Specifically, the worker arranges the vibration transmitter 12 on an inner side of the third support 18I while pushing out both edge portions in the circumferential direction corresponding to a boundary bounded by the slit SL that is provided in the third support 18I in a direction away from each other (FIG. 14C).

Even in a case where the third support 18I is configured by use of a single member as explained above in the ninth modification, the same effects similar to those described above in the embodiment and the eighth modification are provided.

With the ultrasound treatment tool according to the disclosure, it is possible to ensure airtightness and watertightness while improving a cost reduction.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound treatment tool comprising:
a vibration transmitter configured to transmit ultrasound vibration from a proximal end toward a distal end;
a tube into which the vibration transmitter is inserted;
a rigid part that is made of a resin material that is harder than the tube, the rigid part being provided on an outer peripheral surface of the vibration transmitter at a node position of the ultrasound vibration, the rigid part being in contact with an inner peripheral surface of the tube; and
a pipe configured to cover an outer peripheral surface of the tube, wherein
the tube is expanded by the rigid part to be in contact with an inner peripheral surface of the pipe.

2. The ultrasound treatment tool according to claim 1, wherein
the tube is deformable and compressed between the pipe and the rigid part,
a thickness dimension of the tube at a first position at which the tube is compressed between the pipe and the rigid part is smaller than the thickness dimension of the tube at a second position other than the first position.

3. The ultrasound treatment tool according to claim 1, wherein the rigid part is provided at the node position that is located closest to the proximal end from among node positions of the ultrasound vibration on the outer peripheral surface of the vibration transmitter.

4. The ultrasound treatment tool according to claim 1, wherein the rigid part has a configuration in which a first rigid part and a second rigid part are combined.

5. The ultrasound treatment tool according to claim 1, wherein the rigid part has a circular ring shape, and includes a slit that extends from one end toward another end of the rigid part in a longitudinal direction of the vibration transmitter.

6. The ultrasound treatment tool according to claim 1, wherein the tube is made of a resin material that is perfluoroethylene propylene copolymer.

7. The ultrasound treatment tool according to claim 1, wherein the rigid part is made of a resin material that is one of polytetrafluoroethylene, polyether ether ketone, polyphenylsulfone, and polyoxymethylene.

8. The ultrasound treatment tool according to claim 1, wherein
the rigid part includes
a small diameter portion that has a circular ring shape, and
a large diameter portion that has a circular ring shape and has an outer diameter dimension larger than an outer diameter dimension of the small diameter portion,
the tube is deformable and compressed between the pipe and the small diameter portion of the rigid part,
a thickness dimension of the tube at a third position at which the tube is compressed between the pipe and the small diameter portion is smaller than the thickness dimension of the tube at a fourth position other than the third position.

9. The ultrasound treatment tool according to claim 8, wherein
the rigid part further includes a stepped portion between the small diameter portion and the large diameter portion,
an end portion of the tube on a proximal end side is in contact with the stepped portion to be positioned in a direction along a central axis of the tube.

10. The ultrasound treatment tool according to claim 1, wherein the rigid part has a circular ring shape, and includes a tapered portion whose outer diameter dimension changes along a longitudinal direction of the vibration transmitter.

11. The ultrasound treatment tool according to claim 10, wherein the tapered portion includes a first tapered portion whose outer diameter dimension gradually decreases toward the distal end.

12. The ultrasound treatment tool according to claim 10, wherein the tapered portion includes a second tapered portion whose outer diameter dimension gradually decreases toward the proximal end.

13. The ultrasound treatment tool according to claim 11, wherein the tapered portion further includes a second tapered portion whose outer diameter dimension gradually decreases toward the proximal end.

14. The ultrasound treatment tool according to claim 1, further comprising
a jaw configured to be opened and closed with respect to the vibration transmitter,
wherein an electrode for applying high frequency current between the vibration transmitter and the jaw is provided in each of the vibration transmitter and the jaw.

15. The ultrasound treatment tool according to claim 14, wherein an operation handle for opening and closing the jaw with respect to the vibration transmitter in accordance with an operation of a user is provided on a proximal end side of the pipe.

16. The ultrasound treatment tool according to claim 1, wherein the tube is expanded by the rigid part toward the proximal end to be in contact with the inner peripheral surface of the pipe at a position away from the rigid part toward the proximal end in a longitudinal direction of the vibration transmitter.

17. The ultrasound treatment tool according to claim 1, wherein the tube is expanded by the rigid part toward the distal end to be in contact with the inner peripheral surface of the pipe at a position away from the rigid part toward the distal end in a longitudinal direction of the vibration transmitter.

18. The ultrasound treatment tool according to claim 1, wherein
the rigid part is provided with a turn over portion configured to turn up the tube, and
an end portion of the tube that has been turned up by the turn over portion is contact with the inner peripheral surface of the pipe.
